(19) United States
(12) Patent Application Publication
(10) Pub. No.: US 2022/0080146 A1
(43) Pub. Date: Mar. 17, 2022

(54) Respiratory Auxiliary Apparatus

(71) Applicant: Tex-Ray Industrial Co., Ltd., Tainan City (TW)

(72) Inventors: Wan-Kuei Yao, Taoyuan City (TW); Anderson Kenny, New Taipei City (TW); Dyson Philip, New Taipei City (TW)

(73) Assignee: Tex-Ray Industrial Co., Ltd.

(21) Appl. No.: 16/949,364

(22) Filed: Oct. 27, 2020

(30) Foreign Application Priority Data
Sep. 14, 2020 (TW) 109131533

(51) Int. Cl. A61M 16/06; A61M 16/20

(52) U.S. Cl. CPC A61M 16/0666; A61M 2202/0225; A61M 16/208

(57) ABSTRACT

A respiratory auxiliary apparatus is provided, having a nozzle tube formed axially in an air outlet channel of a mask. The air outlet end of the nozzle tube facing the air outlet one-way valve of the air outlet channel gradually reduces the diameter. Vertically corresponding to the center axis of the outlet end, at least one through hole is provided between the side wall of the outlet channel and the side wall of the nozzle tube to communicate the outside of the outlet channel with the inside of the nozzle tube. When the exhaled air passes through the nozzle tube, the air from the outside of the outlet channel is sucked into the nozzle tube to increase the airflow accordingly to the Bernoulli's principle, and enough force is generated when the airflow blows from the nozzle tube to push open the one-way valve to discharge the exhaled carbon dioxide.

### CROSS-REFERENCE TO RELATED APPLICATION

This application claims the priority of Taiwanese patent application No. 109131533, filed on Sep. 14, 2020, which is incorporated herewith by reference.

### BACKGROUND OF THE INVENTION

### 1. Field of the Invention

The present invention relates generally to a medical equipment, and more particularly, to a respiratory auxiliary apparatus for treating respiratory symptoms.

### 2. The Prior Arts

Respiratory auxiliary apparatus are respiratory equipment commonly used in the treatment of respiratory symptoms. Common forms include mask type, head-mounted, nasal plug type, and so on, wherein, general mask-type and head-mounted respiratory auxiliary apparatus includes a mask that covers the patient's nose. The mask is equipped with an air inlet pipe and an air outlet pipe. The air inlet channel of the air inlet pipe is equipped with an air inlet one-way valve, and the air outlet channel of the air outlet pipe is equipped with an air outlet one-way valve. The inlet pipe is connected to the oxygen supply machine through a hose. When the patient wears a respiratory auxiliary device for inhalation, the inhalation creates a negative pressure in the mask, which causes the air inlet one-way valve to open and the air outlet one-way valve to close, so that the oxygen supplied by the oxygen supply machine passes through the air inlet one-way valve and enters the mask but does not exit the air outlet pipe, and the patient can inhale the oxygen. When exhaling, the air inlet one-way valve is closed and the air outlet one-way valve is opened due to the positive pressure formed by the exhaled air in the mask. Therefore, the exhaled carbon dioxide can only be discharged from the air outlet pipe through the air outlet one-way valve and cannot be returned to the oxygen supply machine through the air inlet one-way valve.

As mentioned above, the conventional respiratory auxiliary device must use the airflow force of the patient's exhalation to push open the air outlet one-way valve when expelling the exhaled carbon dioxide. However, because the patient's physical strength is usually not as good as a normal healthy person, it is possible that the airflow force of the patient's exhalation is insufficient to push open the one-way valve of the outlet air, causing carbon dioxide to accumulate in the mask. The patient is likely to have suffer sequelae due to the high concentration of carbon dioxide in the mask.

In addition, “Continuous Positive Airway Pressure (CPAP)” is also used in the treatment of respiratory symptoms, especially in the treatment of obstructive sleep apnea (OSA). In general, the conventional CPAP outputs air when the patient inhales, and supplies the air to the user at a constant pressure to increase the pressure in the patient's respiratory tract to deliver the air into the patient's lungs. On the other hand, when the patient exhales, the air output is stopped, and the carbon dioxide exhaled by the patient is discharged through the exhaust hole on the mask to prevent the carbon dioxide concentration in the mask from becoming too high. However, since the patient does not breathe at a fixed pressure from beginning to end during sleep, when the patient uses a general CPAP during sleep, the patient often has trouble breathing, so another person must control, in accordance with the patient's condition, the CPAP to provide pressure when the patient inhales, which is inconvenient.

Furthermore, the conventional CPAP has an exhaust hole that will cause the wind pressure to decrease during inhalation, and increase the wind pressure when the patient exhales, thereby increasing the difficulty of wind pressure control. Although it is known that the amount of air supplied by the machine can be adjusted by detecting the patient's breathing condition, however, when the patient's breathing condition changes rapidly, the machine must rapidly change accordingly, which not only accelerates the wear of the machine, but also produces excessive noise.

### SUMMARY OF THE INVENTION

A primary objective of the present invention is to solve the problem of conventional respiratory auxiliary apparatus or CPAP, which are likely to cause carbon dioxide accumulation due to insufficient airflow force of the exhalation of weak patients, so that the one-way valve cannot be opened smoothly, which may cause further sequelae and health problems.

The technical means of the respiratory auxiliary apparatus of the present invention includes a mask, an air inlet channel and an air outlet channel communicating with the inner space of the mask; the air inlet channel is provided with an air inlet one-way valve, and the air outlet channel is provided with an air outlet one-way air valve; wherein a nozzle tube is formed axially in the air outlet channel, and the outlet end of the nozzle tube facing the air outlet one-way air valve gradually reduces the diameter; at a position vertically corresponding to the center axis of the air outlet end, at least one through hole is provided between a side wall of the air outlet channel and a side wall of the nozzle tube to communicate the outside of the air outlet channel and the inside of the nozzle tube. With the structure, when the user's exhaled air passes through the nozzle tube, according to the Bernoulli principle, the air outside the outlet channel is drawn into the nozzle tube to increase the air flow, so that sufficient force is generated when the airflow blows out of the nozzle tube to push open the air outlet one-way valve to discharge the exhaled carbon dioxide to avoid the accumulation of carbon dioxide inside the mask.

In an embodiment, the air outlet end of the nozzle tube may be flat. By reducing the diameter of the flat-shaped outlet end, the flow rate and pressure of the exhaled air flow through the outlet end are increased, thereby having sufficient force to push open the one-way valve in the air outlet channel.

In another embodiment, the outlet end of the nozzle tube may be conic. By reducing the diameter of the cone-shaped outlet end, the flow rate and pressure of the exhaled air flow through the outlet end are increased, thereby having sufficient force to push open the one-way valve in the air outlet channel.

In an embodiment, an air inlet pipe is integrally formed with the mask, and the air inlet channel is formed in the air inlet pipe.

In an embodiment, an air outlet pipe is integrally formed with the mask, and the air outlet channel is formed in the air outlet pipe.

Through the aforementioned structure of the respiratory auxiliary apparatus of the present invention, it is not only suitable for general face-mask or head-mounted respiratory auxiliary apparatus, but also applicable to CPAP, and even other respiratory auxiliary apparatus type. With a simple structure, the present invention enlarges the airflow force exhaled by the frail patient, so that the one-way valve for exhalation can be opened smoothly to completely discharge the exhaled carbon dioxide out of the mask, avoiding the adverse sequelae and health issues of excessive carbon dioxide concentration.

### BRIEF DESCRIPTION OF THE DRAWINGS

The present invention will be apparent to those skilled in the art by reading the following detailed description of a preferred embodiment thereof, with reference to the attached drawings, in which:

FIG. 1 is a schematic view illustrating the appearance and structure of the respiratory auxiliary apparatus according to an embodiment of the present invention;

FIG. 2 is a schematic view illustrating the state of the respiratory auxiliary apparatus according to an embodiment of the present invention when worn by a user;

FIG. 3 is a schematic view illustrating the cross-section along the 3-3 direction in FIG. 1;

FIG. 4 is a schematic view illustrating the cross-section along the 4-4 direction in FIG. 1;

FIG. 5 is a schematic view illustrating the state of inhaling the outside air flow when the air flow is blown out of the nozzle tube according to an embodiment of the present invention; and

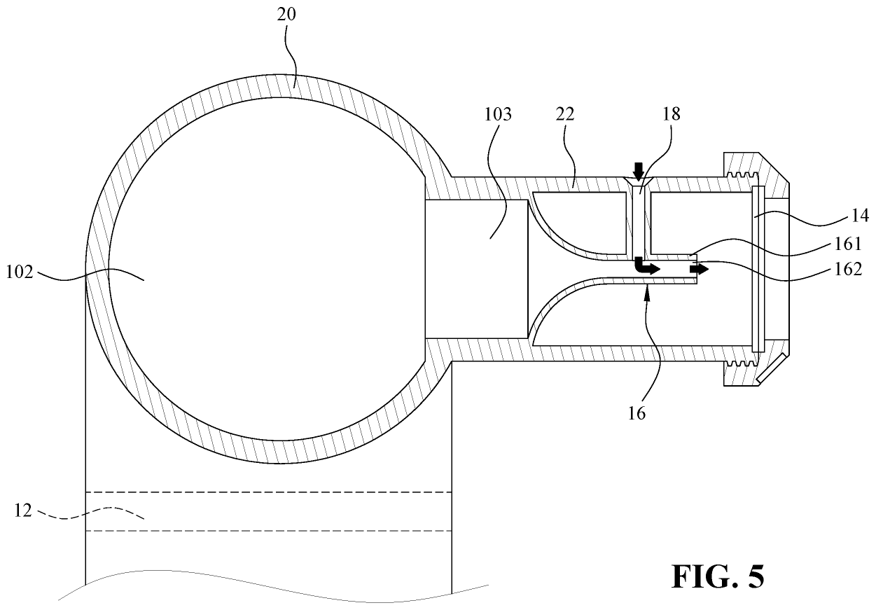

FIG. 6 is a cross-sectional view illustrating another embodiment of the nozzle tube according to the present invention.

### DETAILED DESCRIPTION OF THE PREFERRED EMBODIMENT

The accompanying drawings are included to provide a further understanding of the invention, and are incorporated in and constitute a part of this specification. The drawings illustrate embodiments of the invention and, together with the description, serve to explain the principles of the invention.

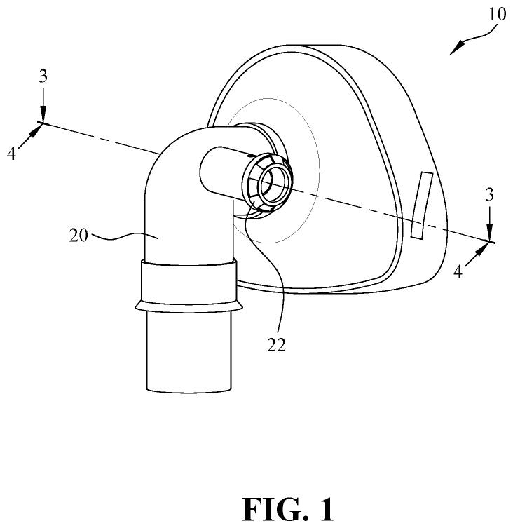

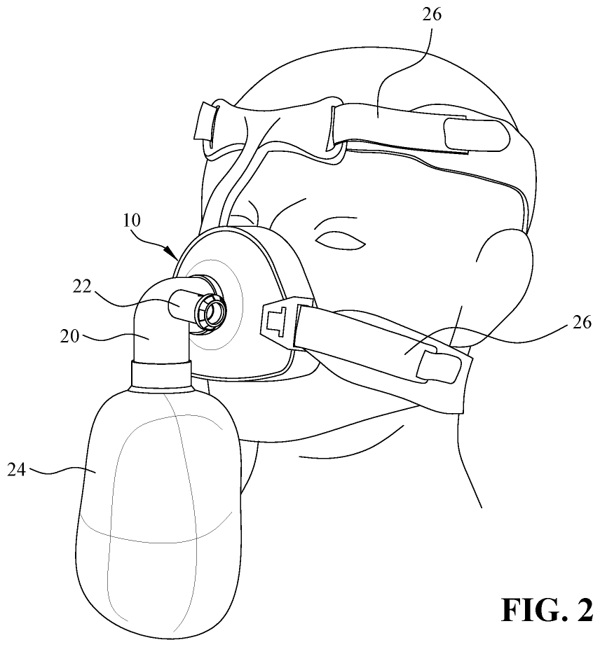

Referring to FIG. 1, a preferred embodiment of the respiratory auxiliary apparatus provided by the present invention includes a mask 10, one side of the mask 10 forms a recessed inner space 101, and the outline of the mask 10 can be formed into an approximate triangle shape to be worn over the mouth and nose of the human face. Elastic bands 26 can be arranged around the periphery of the mask 10 to be worn on the head comfortably and ergonomically (as shown in FIG. 2).

The mask 10 is provided with an air inlet channel 102 and an air outlet channel 103 communicating with the inner space 101. An air inlet one-way valve 12 is provided in the air inlet channel 102, and an air outlet one-way valve 14 is provided in the air outlet channel 103; wherein, the air inlet one-way valve 12 can only be opened in a single direction so that the air can only flow from the air inlet channel 102 to the direction of the inner space 101, but the air in the inner space 101 cannot pass the air inlet one-way valve 12 to outflow. The air outlet one-way valve 14 can only be opened in a single direction so that the air can only flow from the inner space 101 through the air outlet channel 103, but external air cannot flow into the inner space 101 through the air outlet one-way valve 14. The air inlet one-way valve 12 and the air outlet one-way valve 14 may have the same structure, but are arranged in opposite directions to control the unidirectional airflow. The air inlet one-way valve 12 and the air outlet one-way valve 14 are all conventional components and are not technical features claimed by the present invention. Therefore, only their positions are simply shown in the drawings, and the detailed structure is omitted.

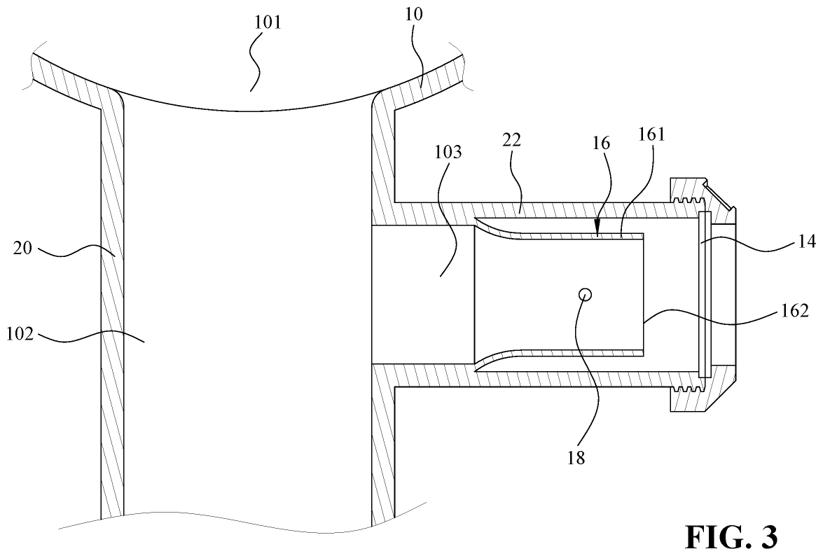

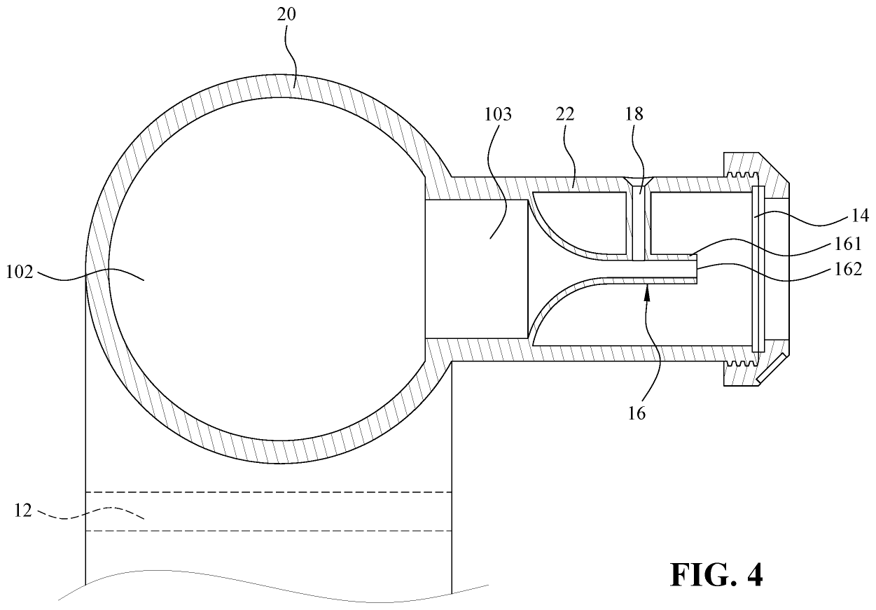

As shown in FIGS. 3 and 4, the present invention further forms integrally a nozzle tube 16 axially parallel in the air outlet channel 103 of the mask 10, and the air outlet end 161 of the nozzle tube 16 facing the air outlet one-way valve 14 is gradually reduced in diameter to form an air outlet mouth 162. At a position vertically corresponding to the central axis of the air outlet end 161, at least one through hole 18 is provided between the side wall of the air outlet channel 103 and the side wall of the nozzle tube 16 to communicate the outside of the air outlet channel 103 with the inside the nozzle tube 16. More specifically, the air outlet end 161 of the nozzle tube 16 may be formed into a flat shape, so that the air outlet mouth 162 of the air outlet end 161 is substantially straight or narrow and rectangular. Accordingly, the inner diameter of the nozzle tube 16 is larger than the air outlet mouth 162, so when the air flow through the nozzle tube 16 out of the air outlet mouth 162, the speed will increase due to the limitation of space reduction, and according to Bernoulli's law, the lateral pressure in the radial direction of the air flow will be reduced when the air passes through the nozzle tube 16 quickly. Therefore, the outside air is introduced into the nozzle pipe 16 through the through hole 18 to increase the air flow.

In addition, in the present invention, an air inlet pipe 20 and an air outlet pipe 22 can be integrally formed at different positions of the mask 10, and the air inlet channel 102 is formed in the air inlet pipe 20, and the air outlet channel 103 is formed In the outlet pipe 22.

When in use, the respiratory auxiliary apparatus of the present invention is can be worn over the user's head in conjunction with the elastic bands 26 arranged on the periphery of the mask 10, so that the mask 10 covers the mouth and nose. Wherein, the air inlet pipe 20 is connected to an oxygen supply machine (not shown in the figure) through a hose, or directly to an oxygen bag 24, as shown in FIG. 2. When the user inhales, the inhalation creates a negative pressure in the mask 10 so that the air inlet one-way valve 12 is opened and the air outlet one-way valve 14 is closed. As a result, the oxygen supplied by the oxygen supply machine or oxygen bag 24 passes through the air inlet one-way valve 12 to enter the inner space 101 of the mask 10 but does not exit the air outlet channel 103, and the user can inhale oxygen. When exhaling, the inner space 101 of the mask 10 closes the air inlet one-way valve 12 and opens the air outlet one-way valve 14 due to the positive pressure formed by the exhaled air. At this time, the exhaled carbon dioxide can only pass through the air outlet one-way valve 14 to exit to the air outlet channel 103 and cannot flow back to the oxygen supply machine or the oxygen bag 24 through the air inlet one-way valve 12. As the patient's physical strength is usually frail, the airflow generated by the exhalation may be weak and the airflow is insufficient. Therefore, when the weaker and smaller amount of airflow passes through the nozzle tube 16, the outside air will pass through the through hole 18 and is introduced into the nozzle tube 16 to increase the air flow rate. The increased air flow rate increases the air flow speed when the air flows from the reduced-diameter air outlet mouth 162, so that the blown air flow can push open the air outlet one-way valve 14 to allow carbon dioxide to be discharged to the outside to avoid too high concentration of carbon dioxide accumulated in the space inside the mask 10 to cause subsequent effects on health.

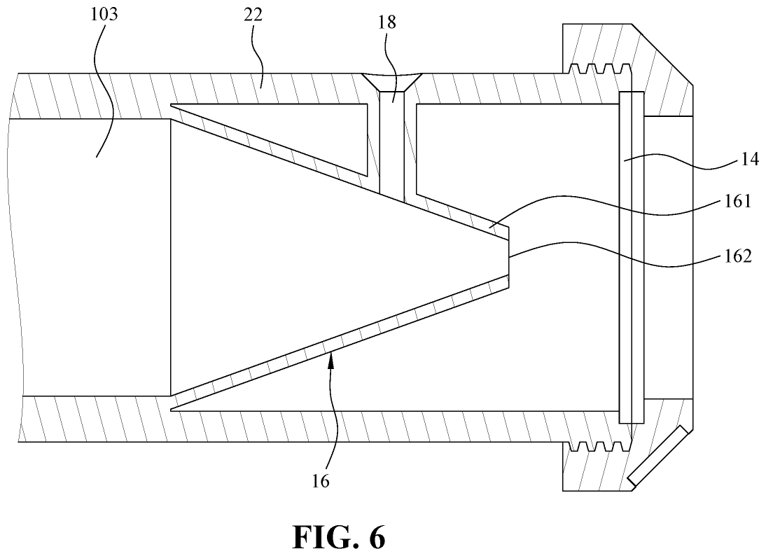

FIG. 6 is a schematic cross-sectional view showing a second embodiment of the nozzle tube 16 of the present invention. In this second embodiment, the air outlet end 161 of the nozzle tube 16 is formed into a tapered shape, such as a cone shape or a pyramid shape. The air outlet mouth of the air outlet 161 may be circular or polygonal. The structure of the nozzle tube 16 of the second embodiment also has the effect of increasing the air flow rate and increasing the air flow velocity, so as to ensure that the air flow blown by the frail patient can push open the air outlet one-way air valve 14 to completely discharge the exhaled carbon dioxide.

Although the present invention has been described with reference to the preferred embodiments thereof, it is apparent to those skilled in the art that a variety of modifications and changes may be made without departing from the scope of the present invention which is intended to be defined by the appended claims.

What is claimed is:

1. A respiratory auxiliary apparatus, comprising: a mask, having an air inlet channel and an air outlet channel communicating with an inner space of the mask; the air inlet channel being provided with an air inlet one-way valve, and the air outlet channel being provided with an air outlet one-way air valve;
wherein a nozzle tube being formed axially in parallel in the air outlet channel, and the outlet end of the nozzle tube facing the air outlet one-way air valve gradually reducing the diameter; at a position vertically corresponding to the center axis of the air outlet end, at least one through hole being provided between a side wall of the air outlet channel and a side wall of the nozzle tube to communicate the outside of the air outlet channel and the inside of the nozzle tube.

2. The respiratory auxiliary apparatus according to claim 1, wherein the outlet end of the nozzle tube is flat.

3. The respiratory auxiliary apparatus according to claim 1, wherein the outlet end of the nozzle tube is conic.

4. The respiratory auxiliary apparatus according to claim 1, wherein an air inlet pipe is integrally formed with the mask, and the air inlet channel is formed in the air inlet pipe.

5. The respiratory auxiliary apparatus according to claim 1, wherein an air outlet pipe is integrally formed with the mask, and the air outlet channel is formed in the air outlet pipe.
